(19) United States
(12) Patent Application Publication
(10) Pub. No.: US 2023/0242702 A1
(43) Pub. Date: Aug. 3, 2023

(54) METHOD FOR OBTAINING BIOSOURCED POLYEPOXIDES WITH IMPROVED PROPERTIES

(71) Applicant: ROQUETTE FRERES, LESTREM (FR)

(72) Inventors: Théodore VANBESIEN, ARMENTIERES (FR); Hélène AMEDRO, BETHUNE (FR); Audrey SAHUT, LESTREM (FR); René SAINT-LOUP, LOMME (FR)

(21) Appl. No.: 18/002,790

(22) PCT Filed: Jun. 25, 2021
(86) PCT No.: PCT/EP2021/025235

§ 371 Date: Dec. 21, 2022

(30) Foreign Application Priority Data
Jun. 30, 2020 (FR) FR2006915

(51) Int. Cl. C08G 59/18; C08G 59/50; C08G 59/68; C07D 303/18

(52) U.S. Cl. CPC C08G 59/184; C08G 59/5026; C08G 59/686; C07D 303/18; C08G 2650/36

(57) ABSTRACT

The invention relates to a method for preparing an epoxy prepolymer composition comprising glycidyl ethers, said method comprising the following steps:
a) placing a dianhydrohexitol into contact with another alcohol so as to obtain a composition of alcohols;
b) reacting the composition of alcohols obtained in step a) with an epihalohydrin so as to obtain a reaction mixture comprising glycidyl ethers;
c) recovering the epoxy prepolymer composition comprising glycidyl ethers from the reaction mixture obtained at the end of step b);
   The invention also relates to an epoxy prepolymer composition that can be obtained by said method, a curable composition comprising said epoxy prepolymer composition, a polyepoxide obtained by curing said curable composition and a composite material, coating or adhesive comprising said polyepoxide.

### TECHNICAL FIELD

The present disclosure relates to the field of polyepoxides and in particular relates to a method for preparing epoxy prepolymers comprising glycidyl ethers of dianhydrohexitol units, as well as glycidyl ethers of other alcohol units.

### PRIOR ART

Polyepoxides, also called epoxide polymers, or commonly called “epoxy”, are widely used, equally as a surface material, for example, for manufacturing adhesives or coatings, and as structural materials, for example, as a matrix of composite materials.

Polyepoxides are obtained by curing curable compositions comprising epoxy prepolymers.

For the purposes of the present invention, an epoxy prepolymer is a mixture of molecules comprising epoxide functions capable of undergoing a subsequent polymerization resulting in the provision of a polyepoxide. The epoxy prepolymers may or may not comprise an oligomeric fraction. They may or may not comprise a polymeric fraction.

Most of the curable compositions comprising epoxy prepolymers that are especially used for manufacturing adhesives, coatings, or matrices for composite materials contain, besides epoxy prepolymers, at least one curing agent and/or at least one accelerating agent.

When curing the curable composition comprising epoxy prepolymers, chemical bonds are formed between molecules of the epoxy prepolymer and/or between the epoxy prepolymer and a curing agent by reactions for opening the epoxide functions of the epoxy prepolymer. This results in the formation of a three-dimensional macromolecular network.

The term “accelerating agent” is understood to mean compounds allowing the homopolymerization reaction between two epoxide functions or the reaction between an epoxide function and the curing agent to be catalyzed. Lewis acids, Lewis bases and photoinitiators are examples thereof.

The term “curing agent” is understood to mean any compound different from the epoxy prepolymers allowing a three-dimensional network to be formed by reacting with the epoxide functions of said prepolymers. The amines, amidoamines, Mannich bases, organic acids (including polyesters terminated by carboxylic functions), organic acid anhydrides, latent curing agents (of the cyanamide, imidazole type, etc.) are examples thereof.

In single-component curable compositions, accelerating agents and/or curing agents are directly incorporated into the epoxy prepolymer composition: reference is made to 1K systems. In two-component curable compositions, the accelerating agent and/or the curing agent is/are packaged separately from the epoxy prepolymer composition and mixing only occurs when shaping of the curable composition is applied: reference is made to 2K systems.

Curable compositions comprising epoxy prepolymers may also contain organic or inorganic fillers (silica, sand, aluminum oxide, talc, calcium carbonate, etc.), pigments, plasticizers, stabilizers, thixotropic agents.

Bisphenol A diglycidyl ether (BADGE), of formula (i), is a chemical compound that is currently very widely used as an epoxy prepolymer.

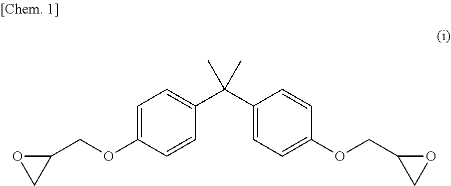 [SMILES: CC(C)(c1ccc(OCC2CO2)cc1)c1ccc(OCC2CO2)cc1]

BADGE is a product obtained from petroleum, which is a disadvantage within a context of increasing prices and/or scarcity of petroleum resources.

Furthermore, bisphenol A is currently acknowledged as being and endocrine disruptor.

This potentially makes the handling of epoxy prepolymers based on bisphenol A or contact with the polyepoxides obtained from BADGE potentially hazardous to health.

For some years it has been known that BADGE may be replaced by mixtures containing isosorbide diglycidylether, which is a biosourced product, the structure of which is depicted below (formula (ii)).

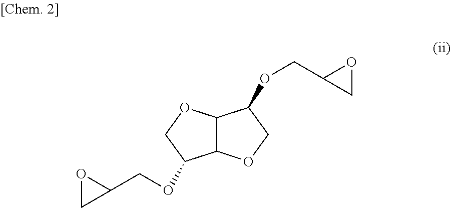 [SMILES: C1OC1CO[C@H]1COC2C1OC[C@H]2OCC1CO1]

This compound, which belongs to the more general class of dianhydrohexitol diglycidyl ethers, is currently widely known and described in literature, as is its synthesis methods. For example, documents U.S. Pat. Nos. 3,272,845, 4,770,871, WO2008/147472, WO2008/147473, U.S. Pat. No. 3,041,300, WO2012/157832 and WO2015/110758 can be cited, which disclose methods for synthesizing dianhydrohexitol diglycidyl ethers.

When any one of the methods described in the aforementioned documents is implemented, an epoxy prepolymer composition containing, in addition to dianhydrohexitol diglycidyl ether, dianhydrohexitol monoglycidyl ether and oligomers comprising dianhydrohexitol and glyceryl units is actually obtained, These oligomers may include one or more glycidyl ether group(s) borne by dianhydrohexitol units and/or glyceryl units.

In the present application, “epoxy prepolymer based on an alcohol” is referred to as an epoxy prepolymer in which the epoxide functions are essentially included in glycidyl ether groups, and in which the glycidyl ether groups are essentially borne by units of said alcohol or glyceryl units that themselves are bonded to units of said alcohol.

For example, in an isosorbide-based epoxy prepolymer, the glycidyl ether groups are essentially borne by isosorbide units and glyceryl units bonded to isosorbide units. The glycidyl ether groups are thus, for example, in the form of isosorbide mono- or di-glycidyl ether or in the form of glycidyl ether-isosorbide- . . . units in oligomers.

Thus, the epoxy prepolymer compositions obtained by implementing the methods described in the aforementioned documents are epoxy prepolymers based on a dianhydrohexitol or based on isosorbide.

In the present application, “polyepoxide based on an alcohol” refers to a polyepoxide obtained by curing an epoxy prepolymer based on said alcohol.

The presence of monoglycidyl ether compounds and/or oligomers in addition to diglycidyl ether compounds in an epoxy prepolymer based on a diol reduces the crosslinking density in the three-dimensional macromolecular network obtained by curing a curable composition comprising said epoxy prepolymer relative to what would be obtained if the curable composition included the diglycidyl ether of said pure diol as an epoxy prepolymer.

This crosslinking density is to be connected to the glass transition temperature (Tg) of the polyepoxide. A high crosslinking density allows a material to be obtained with a higher glass transition temperature (Tg) and that is more chemically and mechanically resistant.

The presence of oligomers and/or mono-glycidyl ethers in an epoxy prepolymer may be directly connected to the epoxy equivalent weight (EEW), defined as the mass of epoxy prepolymer containing an equivalent of glycidyl ether groups For example, pure isosorbide diglycidyl ether (formula ii), which has a molecular weight of 258 g/mol and which contains 2 glycidyl ether groups, has an epoxy equivalent of 129 g/eq.

In an epoxy prepolymer based on a diol, the EEW is minimal if said epoxy prepolymer is diglycidyl ether of said pure diol. The EEW of said epoxy prepolymer increases when the content of oligomer and/or of mono-glycidyl ether of said diol increases in said epoxy prepolymer.

Polyepoxides were prepared from isosorbide diglycidyl ether-based epoxy prepolymers.

However, it is still difficult to obtain biosourced polyepoxides having equivalent performance capabilities to the polyepoxides obtained from petroleum-sourced compounds such as BADGE.

Document US2015/0353676 A1 especially describes isosorbide-based polyepoxides and, as a curing agent, cis-4-cyclohexene-1,2-dicarboxylic acid.

Document US2018/0230261 A1 describes isosorbide-based polyepoxides and a polyamide as a curing agent.

Document WO2015/110758 A1 describes isosorbide-based polyepoxides and isophorone diamine as a curing agent. These polyepoxides have glass transition temperatures of the order of 95-100° C.

Similarly, documents US2017/0253692 and JP2014189713 describe isosorbide-based polyepoxides comprising various curing agents.

A recurring problem encountered until now with isosorbide-based polyepoxides is their significant water uptake, in other words, the water molecules easily diffuse into the three-dimensional macromolecular network of the polyepoxide. Thus, in the presence of moisture or liquid water, these polyepoxides tend to fill with water, which especially results in the polyepoxide plasticizing (reduction of its glass transition temperature), causing it to swell and reducing its cohesion or adhesion properties.

Therefore, significant water uptake is not compatible with many applications of polyepoxides (adhesives, matrix for composite materials, especially), which is currently one of the main hindrances to a broad use of dianhydrohexitol-based polyepoxides.

One solution to this problem, developed by the Applicant company, consists in preparing polyepoxides from mixtures between epoxy prepolymers based on dianhydrohexitols and epoxy prepolymers based on other alcohols.

The objective is to combine the properties of polyepoxides based on dianhydrohexitols and the properties of polyepoxides based on other alcohols, while compensating for their respective defects.

Continuing its research, the Applicant company has nevertheless found that if the dianhydrohexitol and the other alcohol are mixed before the step of synthesizing the epoxy prepolymer, better properties are obtained, especially lower water uptake properties for the resulting polyepoxide, than for the polyepoxide obtained from the mixture of the epoxy prepolymers synthesized separately from the dianhydrohexitol and from the other alcohol. This surprising effect is the subject matter of the present invention.

### SUMMARY

The present disclosure relates to the preparation of epoxy prepolymers comprising dianhydrohexitol glycidyl ethers allowing polyepoxides to be obtained with improved water uptake.

A method for preparing an epoxy prepolymer composition comprising glycidyl ethers is thus proposed, said method comprising the following steps:

a. Placing a dianhydrohexitol into contact with another alcohol so as to obtain a composition of alcohols;
b. Reacting the composition of alcohols obtained in step a) with an epihalohydrin so as to obtain a reaction mixture comprising glycidyl ethers;
c. Recovering the epoxy prepolymer composition comprising glycidyl ethers from the reaction mixture obtained at the end of step b).

According to another aspect, an epoxy prepolymer composition is proposed that can be obtained by the method according to the invention.

According to another aspect, a curable composition is proposed comprising an epoxy prepolymer composition according to the invention, characterized in that it further comprises at least one accelerating agent and/or at least one curing agent.

According to another aspect, a polyepoxide is proposed, obtained by curing a curable composition according to the invention.

According to another aspect, a composite material, a coating or an adhesive comprising a polyepoxide according to the invention is proposed.

Further features and benefits of the present invention will become apparent from reading the following detailed description.

### DETAILED DESCRIPTION

In the present patent application, the expression “comprised between . . . and . . . ” should be understood to include the limit values.

A method is proposed for preparing an epoxy prepolymer composition comprising glycidyl ethers, said method comprising the following steps:

a) Placing a dianhydrohexitol into contact with another alcohol so as to obtain a composition of alcohols;
b) Reacting the composition of alcohols obtained in step a) with an epihalohydrin so as to obtain a reaction mixture comprising glycidyl ethers;
c) Recovering the epoxy prepolymer composition comprising glycidyl ethers from the reaction mixture obtained at the end of step b).

The first step of the method according to the invention (step a) therefore consists in placing a dianhydrohexitol into contact with another alcohol.

The dianhydrohexitol and the other alcohol can be brought into contact by mixing the two compounds, for example, by placing the dianhydrohexitol and the other alcohol in solution or by melting the dianhydrohexitol and the other alcohol in the case where they are miscible in the liquid state, or by mixing these compounds in the solid state, for example, in powder form.

The dianhydrohexitols are heterocyclic compounds obtained by double dehydration of hexitols (iditol, mannitol or sorbitol, for example). They are therefore diols. Among dianhydrohexitols, isohexides correspond to 1,4:3,6-dianhydrohexitols and comprise isosorbide, isoidide and isomannide.

In the method according to the invention, the dianhydrohexitol is preferably an isohexitol, more preferentially selected from isosorbide, isomannide and isoidide, and is most preferentially isosorbide.

In the method according to the invention, step b. of reacting with an epihalohydrin allows the alcohol functions of the dianhydrohexitol and of the other alcohol to be transformed into glycidyl ether groups. In this step, oligomers also may be formed. Among these oligomers, some comprise dianhydrohexitol units or other alcohol units and some comprise a mixture of dianhydrohexitol units and of other alcohol units.

The reaction mixture at the end of step b. therefore contains an epoxy prepolymer composition comprising glycidyl ether groups borne by dianhydrohexitol units and glycidyl ether groups borne by other alcohol units.

Mixing at least one dianhydrohexitol and at least one other alcohol in step a., in other words, prior to step b. of reacting with the epihalohydrin, results in an unexpected effect.

Indeed, the polyepoxides obtained by curing curable compositions comprising epoxy prepolymer compositions obtained by the method according to the invention exhibit properties, for example, water uptake properties, that are improved relative to the properties of polyepoxides obtained by curing comparable curable compositions resulting from mixing a dianhydrohexitol-based epoxy prepolymer and an epoxy prepolymer based on the other alcohol.

Without wishing to limit the scope of the invention to any theory, this unexpected improvement of the polyepoxide may be considered to be related to the presence of oligomers comprising both dianhydrohexitol units and other alcohol units in the epoxy prepolymer composition obtained by the method of the invention.

In the method according to the present invention, the other alcohol brought into contact with a dianhydrohexitol in step a. preferably is not a dianhydrohexitol.

More preferentially, the other alcohol brought into contact with a dianhydrohexitol in step a. is selected from the following alcohols:
Trimethylol ethane,
Trimethylol propane,
Spiroglycol,
Tricyclodecanedimethanol,
Glycerol,
Hexan-1,6-diol,
Cₙ aliphatic diols, where n≥7,
Cyclohexan-1, m-dimethanol, where m=2, 3 or 4,
Furan-p,q-dimethanol, where {p,q}={1,4}, {1,3} or {2,3},
Thiophen-p,q-dimethanol, where {p,q}={1,4}, {1,3} or {2,3},
Isoborneol
Dodecanol, or
Decanol.

According to one embodiment of the method according to the invention, the other alcohol comprises at least two alcohol functions.

If the other alcohol only comprises one alcohol function, it can only be integrated at the end of the chain in oligomers comprising both dianhydrohexitol units and other alcohol units contained in the epoxy prepolymer composition obtained by the method of the invention. If the other alcohol comprises at least two alcohol functions, the epoxy prepolymer composition obtained by the method of the invention may comprise oligomers comprising . . . -dianhydrohexitol-glyceryl-other alcohol-glyceryl units; or . . . -dianhydrohexitol-glyceryl-other alcohol-glycidyl units.

Advantageously, the other alcohol brought into contact with a dianhydrohexitol in step a. of the method according to the invention is selected such that a polyepoxide based on said other alcohol has a lower water uptake than that of a polyepoxide based on said dianhydrohexitol.

In other words, a polyepoxide obtained by a method for curing an epoxy prepolymer composition obtained by means of a method for the etherification of the other alcohol with an epihalohydrin, preferably has a water uptake that is lower than the water uptake, measured according to the same method, of a polyepoxide obtained by the same curing and etherification methods but by replacing the other alcohol with dianhydrohexitol.

In the method according to the invention, the other alcohol is preferably 1,4-cyclohexanedimethanol (CHDM).

In the method according to the invention, the ratio r between the number of moles of the dianhydrohexitol and the sum of the number of moles of the other alcohol and the number of moles of the dianhydrohexitol (r=n_{dianhydrohexitol}/(n_{dianhydrohexitol}+n_{other alcohol})) is preferably comprised between 0.05 and 0.95, more preferably between 0.1 and 0.9.

In the method according to the invention, the epihalohydrin is preferably selected from epibromohydrin, epifluorohydrin, epiiodohydrin and epichlorhydrin, and is, more preferentially, epichlorhydrin.

In the method according to the invention, step b. of reacting with epihalohydrin allows alcohol functions of the dianhydrohexitol and the other alcohol to be transformed into glycidyl ether groups.

It can be carried out according to any method known to a person skilled in the art allowing alcohol functions of the dianhydrohexitol and the other alcohol to be transformed into glycidyl ether groups, for example, the methods described in documents U.S. Pat. Nos. 3,272,845, 4,770,871, WO2008/147472, WO2008/147473, U.S. Pat. No. 3,041,300, WO2012/157832 and WO2015/110758, preferably the method described in document WO2015/110758.

Thus, in the method according to the invention, step b) of reacting the composition of alcohols with the epihalohydrin preferably comprises the following steps:

b1) Placing the composition of alcohols into contact with the epihalohydrin so as to obtain a reaction mixture;
b2) Placing the reaction mixture obtained in step b1) under vacuum so as to obtain a negative pressure comprised between 100 mbar and 1,000 mbar;
b3) Heating the reaction mixture obtained in step b2), while maintaining said negative pressure, at a temperature comprised between 50° C. and 120° C., so as to achieve distillation of the epihalohydrin;
b4) Adding a basic reagent to the reaction mixture obtained in step b3) for a period comprised between 1 hour and 10 hours, while maintaining the reaction mixture at said negative pressure and at said temperature so as to achieve azeotropic distillation of the water-epihalohydrin azeotrope.

Step b1) of placing into contact the composition of alcohols and epihalohydrin is carried out in any device well known to a person skilled in the art, allowing chemical reagents to be brought into contact, and being provided with heating and stirring components. By way of example, it may be a double jacketed reactor. The device in question also must be equipped with a component for producing a partial vacuum and a component for carrying out azeotropic distillation, such as an inverted Dean-Stark assembly surmounted by a condenser.

The epihalohydrin is preferentially introduced in excess relative to the hydroxyl functions of the alcohols present in the composition of alcohols (comprising dianhydrohexitol and the other alcohol). Thus, for 1 mole of hydroxyl functions between 1 and 5 moles of epihalohydrin will preferentially be introduced, and more preferentially approximately 2.5 moles of epihalohydrin.

After this first step of placing into contact (step b1), a partial vacuum is produced in the device using a vacuum pump, with the corresponding negative pressure comprised between 100 mbar and 1,000 mbar (step b2). This means that the pressure in the reactor is equal to the difference between the atmospheric pressure (1,013 mbar) and the negative pressure (between 100 mbar and 1,000 mbar), or a pressure in the reactor comprised between 13 mbar and 913 mbar.

According to one embodiment, step b2 is carried out so as to obtain a negative pressure comprised between 300 and 900 mbar, in particular between 500 and 800 mbar.

During step b3), the mixture is heated between the composition of alcohols and the epihalohydrin at a temperature comprised between 50° C. and 120° C.

Preferably, the setpoint temperature of the heating component of the reactor must be adjusted so as to be at least equal to the boiling temperature of the epihalohydrin that is used, so as to begin the distillation of the epihalohydrin. The boiling temperature that should be taken into account is the boiling temperature of the epihalohydrin at the pressure that prevails in the reactor.

During this first distillation phase, said distillation relates only to epihalohydrin. In addition, only part of the epihalohydrin is distilled. This distilled part can be recovered, for example, on an inverted Dean Stark and optionally reintroduced into the reaction mixture.

By way of example, the epichlorohydrin has a boiling temperature of 116° C. at atmospheric pressure, with this boiling temperature being approximately equal to 80° C. when the pressure in the reactor is 275 mbar (which corresponds to a negative pressure of 738 mbar). Conveniently, the setpoint temperature of the heating component of the reactor will be adjusted to a slightly higher temperature (approximately 30° C. more) than the boiling temperature for the considered epihalohydrin and for the imposed negative pressure.

During step b4), a basic reagent is added to the composition of alcohols/epihalohydrin, for a period comprised between 1 hour and 10 hours.

The amount of basic reagent is preferably the stoichiometric amount relative to the number of hydroxyl functions of the alcohols present in the composition of alcohols. It is nevertheless possible to decide to be slightly in excess relative to this stoichiometry.

The basic reagent is preferably selected from lithium, potassium, calcium or sodium hydroxides, preferably in the form of an aqueous solution, and is more preferably an aqueous sodium hydroxide solution.

The ratio of the number of moles of OH⁻ introduced with the basic reagent to the number of moles of hydroxyl functions of the alcohols present in the composition of alcohols is then preferably comprised between 0.9 and 1.2.

As soon as the basic reagent is introduced (step b4), water is formed by a reaction between the composition of alcohols and the epihalohydrin, just as additional water may be added by introducing the basic reagent in the form of an aqueous solution. The distillation is then an azeotropic distillation, which relates to the water and epihalohydrin mixture. In other words, the water-epihalohydrin azeotrope is distilled. After the distilled azeotrope settles, the water is removed and the epihalohydrin returns to the reaction medium. In the case of an inverted Dean-Stark device, water constitutes the upper phase that is removed, while the epihalohydrin in the lower part is reintroduced into the reaction medium.

The azeotropic distillation is preferably continued until the water is completely removed. Thus, the reaction medium is still heated for a period comprised between 30 minutes and 1 hour after the end of the addition of the basic reagent.

Preferably, a phase-transfer catalyst is added during step b1). It is thus possible to significantly reduce the viscosity of the manufactured products, while maintaining a very high proportion of dianhydrohexitol diglycidyl ether relative to the dianhydrohexitol monoglycidyl ether.

The phase-transfer catalyst is preferably selected from tetraalkylammonium halides, sulfates or hydrogensulfates, and more preferentially from tetrabutylammonium bromide or tetrabutylammonium iodide.

The amount of phase-transfer catalyst is preferably comprised between 0.01 and 5%, more preferentially between 0.1% and 2% and even more preferentially is 1% by weight relative to the total represented by the sum of the masses of the dianhydrohexitol and of the other alcohol. The EEW of the obtained epoxy prepolymer composition can thus be reduced very significantly.

Preferably, step c) comprises a step of filtering the reaction medium obtained at the end of step b) so as to obtain a filtrate comprising the epoxy prepolymer composition. This filtration step allows the salts formed during the reaction between the epihalohydrin and the composition of alcohols to be removed, such as sodium chloride in the case of epichlorohydrin. The salts separated by filtration are preferably washed once again with epihalohydrin. The washing epihalohydrin combined with the first filtrate then constitutes the filtrate comprising the epoxy prepolymer composition.

The filtration step (including washing of the removed salts) is preferably followed by a step of concentrating the filtrate and/or of purifying the filtrate.

The concentration step may allow, for example, unreacted epihalohydrin and/or washing epihalohydrin to be removed. This can occur, for example, by vacuum distillation, for example, in a rotary evaporator and/or a wiped film evaporator device. During this concentration step, the crude product or the epoxy prepolymer composition is, for example, gradually heated up to 140° C. and the pressure is, for example, reduced to 1 mbar (corresponding to a negative pressure of 1,012 mbar).

The purification step can be carried out, for example, by distillation under reduced pressure (pressure <1 mbar corresponding to a negative pressure >1,012 mbar) and can be carried out by means of a wiped surface exchanger in order to separate oligomeric compounds from the dianhydrohexitol diglycidyl ether compounds or from the other alcohol. This step is distinct from that described in the preceding paragraph.

According to another aspect of the present invention, an epoxy prepolymer composition is proposed that can be obtained by the method according to the invention.

The epoxy prepolymer compositions that can be obtained by the method according to the invention have the advantage of having a reduced viscosity relative to dianhydrohexitol-based epoxy prepolymers obtained by the same method but without adding another alcohol to the dianhydrohexitol.

Advantageously, the epoxy prepolymer compositions that can be obtained by the method according to the invention have a Brookfield viscosity, measured at 25° C., of less than 4,000 mPa·s, preferably of less than 1,000 mPa·s, without requiring a purification step by separating the oligomeric compounds from the diglycidyl ether compounds.

Low viscosity facilitates the shaping and processability of the epoxy prepolymers. For example, manufacturing by casting, coating, infusion, impregnation, lamination, injection, pultrusion, or filamentary winding of composite materials comprising a polyepoxide as a matrix is facilitated. Low viscosity also allows for example deposits of thin layers, the use of a spray gun, of a roller to be facilitated when implementing polyepoxides as coatings or adhesives.

Viscosity is measured using a Brookfield DV-II+ type viscometer. The measurement is carried out after stabilization of the medium maintained at 25° C. using a thermostatically controlled water bath. The viscosity measurements are obtained with a torque as a % of the maximum torque of the viscometer comprised between 10 and 100%.

Throughout the present Application, the speed at which the Brookfield viscosity is determined is not indicated. A person skilled in the art indeed knows how to adapt it relative to the choice of the rotor and so as to be placed at a percentage of the maximum torque of the viscometer comprised between 10 and 100%.

Once prepared, the epoxy prepolymer composition according to the invention may be crosslinked to form a cured polyepoxide. It may be preferable to add a curing agent and/or an accelerating agent to initiate or accelerate the crosslinking.

According to another aspect of the present invention, a curable composition is proposed comprising an epoxy prepolymer composition according to the invention, characterized in that it further comprises at least one accelerating agent and/or at least one curing agent.

“Curable composition” is intended to mean a liquid mixture that is capable of polymerizing to form a crosslinked (cured) resin. For example, a curable composition comprising epoxy prepolymers is a liquid mixture capable of polymerizing to form a polyepoxide, which by definition is a crosslinked resin.

Preferably, the curable composition comprises an amine-type curing agent

The amine curing agent may be selected, for example, from:
linear aliphatic diamines, in particular 1,2-diaminomethane, 1,3-diaminopropane, butane-1,4-diamine, pentane-1,5-diamine, 1,6-diaminohexane, or 1,12-diaminododecane,
cyclic aliphatic diamines, in particular isophorone diamine (IPDA), 4,4′-diaminodicyclohexylmethane (PACM), 1,2-diaminocyclohexane (DACH), menthanediamine, or 1,3-bis(aminomethyl)cyclohexane (1,3 BAC),
aromatic diamines, in particular 4,4′-methylenebis(2 aminophenyl)fluorene (BAFL), diethyltoluene diamine (DETDA), dimethyl aminophenyl fluorene (BAFL), diethyltoluene diamine (DETDA), dimethyl thiotoluene diamine (DMTDA), 4,4′-methylenebis(2-ethylaniline) (MOEA), m-xylenediamine, m-phenylenediamine (MPDA), or 4,4′-diaminodiphenylmethane,
triamines, in particular diethlyene triamine (DTA),
tetramines, in particular triethylenetetramine,
pentamines, in particular tetraethylenepentamine,
diamines of fatty acid dimers, in particular Priamine® 1074 by Croda,
polyetheramines, in particular poly(oxypropylene) diamine (Jeffamine® D-230 by Huntsman Petrochemical, LLC), or poly(oxypropylene) triamine (Jeffamine® T-403 by Huntsman Petrochemical, LLC), or
any other polyamine, in particular polyethylene imine (for example, Lupasol® FG from BASF), dipropenediamine, diethylaminopropylamine, N-aminoethylpiperazine, dicyandiamide (Dicy),
or a mixture thereof.

Preferably, the curing agent is isophorone diamine.

The epoxy/amine system formed by the curable composition according to the invention may be stoichiometric or contain an excess of amine functions or an excess of epoxy functions.

The ratio of the number of N—H bonds in the formula of the curing agent (D) to the number of epoxy groups of the epoxy prepolymer composition thus may be comprised between 1:2 and 2:1, in particular between 2:3 and 3:2, more particularly be equal to 1:1 (a stoichiometric mixture).

For example, a primary amine function comprises two N—H bonds. Thus, 4 N—H bonds are counted per molecule of isophorone diamine.

Preferably, in the curable composition according to the invention, the accelerating agent is selected from Lewis acids, tertiary amines or imidazole and derivatives thereof.

According to one embodiment, in the curable composition according to the invention, the accelerating agents and/or curing agents are directly incorporated into the epoxy prepolymer composition. The curable composition according to the invention is then of the single-component type (1K system).

According to one embodiment, in the curable composition according to the invention, the accelerating agents and/or the curing agents are packaged separately from the epoxy prepolymer composition. The curable composition according to the invention is then of the two-component type (2K system).

According to another aspect of the present invention, a polyepoxide is proposed, obtained by curing the curable composition according to the invention.

Curing (that is, crosslinking) of the curable composition according to the invention can occur spontaneously or else require heating or irradiation by UV radiation.

In particular, the curable composition according to the invention may be crosslinked at a temperature comprised between 5° C. and 260° C.

More particularly, the curable composition according to the invention can undergo a curing cycle optionally including a period at ambient temperature followed by one or more heating period(s) at increasing temperatures and comprised between 30° C. and 260° C. For example, the curable composition according to the invention can undergo a curing cycle of 1 hour at 80° C. followed by 2 hours at 180° C.

Preferably, the polyepoxide according to the invention has a glass transition temperature (Tg) greater than or equal to 70° C., in particular comprised between 70° C. and 210° C., more particularly comprised between 90° C. and 200° C.

The glass transition temperature of the polyepoxide according to the invention may be determined by techniques known to a person skilled in the art, especially by differential scanning calorimetry (DSC), for example using a DSC Q20 device in an open crucible with a Heat/Cool/Heat cycle from 0° C. to 200° C. at 10° C./min, or by dynamic mechanical analysis (DMA), for example, using an Anton Paar MCR 501 rheometer equipped with a tension clamp at a temperature regulated from 25° C. to 250° C. at 5° C./min and a frequency of 1 Hz.

Moreover, the polyepoxide according to the invention has a water uptake that is less than or equal to 15%, in particular comprised between 0.1% and 11%, more particularly between 0.5% and 9.5%, preferably between 1% and 9%, more particularly between 1.5% and 8.5%, even more particularly between 2% and 8%.

The water uptake of the polyepoxide is determined by measuring the mass of a sample before and after saturation with water obtained by immersion in water at ambient temperature for a sufficient time. For example, the water uptake of the polyepoxide may be determined on 50 mm×25 mm×2 mm parallelepiped samples immersed in water for 96 hours at ambient temperature, according to the following formula:

$$water uptake ( \% ) = \frac{( ( mass after immersion - dry mass ) )}{( dry mass )} \qquad [ Math . 1 ]$$

According to another aspect of the present invention, a composite, coating or adhesive material is proposed comprising the polyepoxide according to the invention.

The composite materials according to the invention may be composite materials of the polyepoxide/fibers type, the fibers of which especially may be selected from glass fibers, carbon fibers, basalt fibers, plant fibers (flax, hemp).

The composite materials according to the invention may be useful for manufacturing parts with structural performance capabilities, such as, for example, in the automotive field, the nautical field, the aeronautics field or even in the sports and leisure field.

### EXAMPLES

The water uptake of the samples is determined on 50 mm×25 mm×2 mm parallelepiped samples immersed in water for 96 hours at ambient temperature, according to the following formula:

$$water uptake ( \% ) = \frac{( ( mass after immersion - dry mass ) )}{( dry mass )} \qquad [ Math . 2 ]$$

The glass transition temperatures (Tg) are determined by differential scanning calorimetry (DSC) under the following conditions:

### Device: DSC Q20

Between 10 and 20 mg of product is deposited in an open crucible. A Heat/Cool/Heat cycle is carried out from 0° C. to 200° C. at 10° C./min.

Viscosities are measured using a Brookfield DV-II+ rotary viscometer. The measurement is carried out after stabilization of the medium maintained at 25° C. using a thermostatically controlled water bath. The viscosity measurements are obtained with a torque as a % of the maximum torque of the viscometer comprised between 10 and 100%.

The epoxy equivalent weight (EEW) is measured according to standard ISO 3001 or ASTM D1652.

### Example 1: Isosorbide 100% (Comparative Example)

200 g of isosorbide, 633 g of epichlorohydrin (5 molar equivalents relative to the diol) and 2 g of tetraethylammonium bromide (TEAB, 1% by mass relative to the mass of diol) are introduced into a 2.5 L capacity double jacketed reactor, equipped with a stirrer blade and a reverse Dean Stark surmounted by a condenser. The reaction medium is heated (setpoint temperature: 110° C.) under a partial vacuum, ensured by a vane pump, of 275 mbar (which corresponds to a negative pressure of 1,013−275=738 mbar). After distillation of an amount of epichlorohydrin sufficient to fill the reverse Dean-Stark, 230 g of aqueous solution of sodium hydroxide at 50% by mass is introduced over a period of 3 hours using a peristaltic pump. During the addition of sodium sulfate, the distillation of the water-epichlorohydrin azeotrope and the demixing in the Dean-Stark allow the water introduced and formed during the reaction to be removed. Once the addition of sodium sulfate is complete, the medium is allowed to warm and distill until the medium reaches a temperature of 90° C. Once this temperature is reached, heating is stopped and the medium is left to cool at ambient temperature. The medium is then stripped, and the salts formed during the reaction are filtered using a porosity 3 sintered glass. The salt cake is then washed using 150 g of acetone. The filtrate is recovered. The washing solvents and the residual epichlorohydrin are eliminated by distillation under vacuum using a rotary evaporator. 338 g of a yellow homogeneous viscous oil are obtained. The results of the analyses carried out on the obtained epoxy prepolymer are presented in Table 1.

TABLE 1

| | Example 1 | Example 2 | Example 3 |
|---|---|---|---|
| EEW (g/eq.) | 189 | 164 | 169 |
| Isosorbide conversion rate | 100% | 99.3% | 99.6% |
| CHDM conversion Rate | — | 91.1% | 79.6% |
| Viscosity (mPa · s) | 4360 | 175 | 990 |
| Water content (g/100 g) | 0.15 | 0.02 | 0.05 |

### Example 2: Isosorbide 25%/CHDM 75% (Example According to the Invention)

10 g of isosorbide, 29.3 g of CHDM, 126 g of epichlorohydrin (5 molar equivalents relative to the diol) and 400 mg of tetraethylammonium bromide (TEAB, 1% by mass relative to the diol) are introduced into a 500 mL capacity double jacketed reactor, equipped with a stirrer blade and a reverse Dean Stark surmounted by a condenser. The reaction medium is heated (setpoint temperature: 110° C.) under a partial vacuum, ensured by a vane pump, of 275 mbar (which corresponds to a negative pressure of 1,013−275=738 mbar). After distillation of an amount of epichlorohydrin sufficient to fill the reverse Dean-Stark, 45 g of aqueous solution of sodium hydroxide at 50% by mass is introduced over a period of 3 hours using a peristaltic pump. During the addition of sodium sulfate, the distillation of the water-epichlorohydrin azeotrope and the demixing in the Dean-Stark allow the water introduced and formed during the reaction to be removed. Once the addition of sodium sulfate is complete, the medium is allowed to warm and distill until the medium reaches a temperature of 90° C. Once this temperature is reached, heating is stopped and the medium is left to cool at ambient temperature. The medium is then stripped, and the salts formed during the reaction are filtered using a porosity 3 sintered glass. The salt cake is then washed using 50 g of acetone. The filtrate is recovered. The washing solvents and the residual epichlorohydrin are eliminated by distillation under vacuum using a rotary evaporator. 67 g of a yellow homogeneous viscous oil is obtained. The results of the analyses carried out on the obtained epoxy prepolymer are presented in Table 1.

### Example 3: Isosorbide 75%/CHDM 25% (Example According to the Invention)

174.6 g of isosorbide, 58.2 g of CHDM, 644 g of epichlorohydrin (5 molar equivalents relative to the diol) and 2.32 g of tetraethylammonium bromide (TEAB, 1% by mass relative to the mass of diol) are introduced into a 2.5 L capacity double jacketed reactor, equipped with a stirring blade and a reverse Dean Stark surmounted by a condenser. The reaction medium is heated (setpoint temperature: 110° C.) under a partial vacuum, ensured by a vane pump, of 275 mbar (which corresponds to a negative pressure of 1,013−275=738 mbar). After distillation of an amount of epichlorohydrin sufficient to fill the reverse Dean-Stark, 235 g of aqueous solution of sodium hydroxide at 50% by mass is introduced over a period of 3 hours using a peristaltic pump. During the addition of sodium sulfate, the distillation of the water-epichlorohydrin azeotrope and the demixing in the Dean-Stark allow the water introduced and formed during the reaction to be removed. Once the addition of sodium sulfate is complete, the medium is allowed to warm and distill until the medium reaches a temperature of 90° C. Once this temperature is reached, heating is stopped and the medium is left to cool at ambient temperature. The medium is then stripped, and the salts formed during the reaction are filtered using a porosity 3 sintered glass. The salt cake is then washed using 150 g of acetone. The filtrate is recovered. The washing solvents and the residual epichlorohydrin are eliminated by distillation under vacuum using a rotary evaporator. 352 g of a yellow homogeneous viscous oil are obtained. The results of the analyses carried out on the obtained epoxy prepolymer are presented in Table 1.

The products from examples 2 and 3 (respectively isosorbide 25%/CHDM 75% and isosorbide 75%/CHDM 25%) were crosslinked with isophorone diamine (IPDA). Thus

### Example 4

5 grams of the product of Example 2 were mixed with 1.30 g of IPDA before undergoing a curing cycle of 1 hour at 80° C. followed by 2 hours at 180° C.

### Example 5

5 grams of the product of Example 3 were mixed with 1.27 g of IPDA before undergoing a curing cycle of 1 hour at 80° C. followed by 2 hours at 180° C.

The glass transition temperature as well as the water uptake of the crosslinked products thus obtained were measured.

The results are presented in Table 2.

TABLE 2

| | | Tg 2^{nd} |
|---|---|---|
| Polyepoxide | Water uptake | heating (° C.) |
| Example 4 | 2.39% | 70 |
| Example 5 | 10.9% | 98 |
| Example 6 | 3% | 83 |
| Example 7 | 13% | 99 |
| Example 8 | 25% | 110 |

By way of comparison, the crosslinking of isosorbide mixtures of isosorbide/CHDM glycidyl ether was carried out. Thus

### Example 6

5 grams of a composition comprising 25 mol % of isosorbide diglycidyl ether (EEW=189 g/eq) and 75 mol % of diglycidyl ether of CHDM (EEW=159 g/eq) were vigorously mixed for 5 minutes with 1.28 g of IPDA before undergoing a curing cycle of 1 hour at 80° C. followed by 2 hours at 180° C. in an oven

### Example 7

5 grams of a composition comprising 75 mol % of isosorbide diglycidyl ether (EEW=189 g/eq) and 25 mol % of diglycidyl ether of CHDM (EEW=159 g/eq) were vigorously mixed for 5 minutes with 1.17 g of IPDA before undergoing a curing cycle of 1 hour at 80° C. followed by 2 hours at 180° C. in an oven.

The glass transition temperature as well as the water uptake of the crosslinked products thus obtained were measured.

The results are presented in Table 2.

Comparison of the materials according to the invention and of the materials made from epoxy prepolymer mixtures, shows that the first have better water resistance (lower water uptake).

By way of comparison, crosslinking of an isosorbide diglycidyl ether using IPDA was carried out. Thus

### Example 8

5 grams of isosorbide diglycidyl ether (EEW=189 g/eq) were vigorously mixed for 5 minutes with 1.12 g of IPDA before undergoing a curing cycle of 1 hour at 80° C. followed by 2 hours at 180° C. in an oven

Comparing the materials according to the invention and the polyepoxide obtained from isosorbide diglycidyl ether alone shows that the first materials have significantly improved water resistance and reduced thermal resistance.

1. A method for preparing an epoxy prepolymer composition comprising glycidyl ethers, said method comprising the following steps:
a) Placing a dianhydrohexitol into contact with another alcohol so as to obtain a composition of alcohols;
b) Reacting the composition of alcohols obtained in step a) with an epihalohydrin so as to obtain a reaction mixture comprising glycidyl ethers;
c) Recovering the epoxy prepolymer composition comprising glycidyl ethers from the reaction mixture obtained at the end of step b).

2. The method according to claim 1, wherein the dianhydrohexitol is an isohexitol, preferably selected from isosorbide, isomannide or isoidide, and is, more preferably, isosorbide.

3. The method according to claim 1, wherein the other alcohol is selected from the following alcohols:
Trimethylol ethane,
Trimethylol propane,
Spiroglycol,
Tricyclodecanedimethanol,
Glycerol,
Hexan-1,6-diol,
Cₙ aliphatic diols, wherein n≥7,
Cyclohexan-1, m-dimethanol, where m=2, 3 or 4,
Furan-p,q-dimethanol, where {p,q}={1,4}, {1,3} or {2,3},
Thiophen-p,q-dimethanol, where {p,q}={1,4}, {1,3} or {2,3},
Isoborneol,
Dodecanol, or
Decanol.

4. The method according to claim 1, wherein the other alcohol comprises at least two alcohol functions.

5. The method according to claim 1, wherein a polyepoxide obtained by a method for curing an epoxy prepolymer composition obtained by a method for the etherification of the other alcohol with an epihalohydrin, preferably has a water uptake lower than the water uptake, measured according to the same method, of a polyepoxide obtained by the same curing and etherification methods but by replacing the other alcohol with dianhydrohexitol.

6. The method according to claim 1, wherein the epihalohydrin is selected from epibromohydrin, epifluorohydrin, epiiodohydrin, epichlorhydrin, or mixtures thereof, preferably epihalohydrin is epichlorhydrin.

7. The method according to claim 1, wherein step b) of reacting the composition of alcohols with epihalohydrin comprises the following steps:
b1) Placing the composition of alcohols into contact with epihalohydrin so as to obtain a reaction mixture;
b2) Placing the reaction mixture obtained in step b1) under vacuum so as to obtain a negative pressure comprised between 100 mbar and 1,000 mbar;
b3) Heating the reaction mixture obtained in step b2), while maintaining said negative pressure, at a temperature comprised between 50° C. and 120° C., so as to achieve distillation of the epihalohydrate;
b4) Adding a basic reagent to the reaction mixture obtained in step b3) for a period comprised between 1 hour and 10 hours while maintaining the reaction mixture at said negative pressure and at said temperature so as to achieve azeotropic distillation of the water-epihalohydrin azeotrope.

8. The method according to claim 7, wherein the basic reagent is selected from lithium, potassium, calcium or sodium hydroxides, preferably in the form of an aqueous solution, and is more preferably an aqueous sodium hydroxide solution.

9. The method according to claim 7, wherein a phase-transfer catalyst is added during step b1).

10. The method according to claim 9, wherein the phase-transfer catalyst is selected from halides, tetraalkyl ammonium sulfates or hydrogen sulfates, preferably from tetrabutylammonium bromide or tetrabutylammonium iodide.

11. The method according to claim 9, wherein the amount of phase-transfer catalyst is comprised between 0.01 and 5%, preferably between 0.1% and 2%, more preferably is 1% by weight relative to the total represented by the sum of the masses of the dianhydrohexitol and of the other alcohol.

12. The method according to claim 1, wherein step c) comprises a step of filtering the reaction medium obtained at the end of step b) so as to obtain a filtrate comprising the epoxy prepolymer composition, preferably said filtration step is followed by a step of concentrating the filtrate and/or a step of purifying the filtrate.

13. An epoxy prepolymer composition that can be obtained by the method according to claim 1.

14. A curable composition comprising the epoxy prepolymer composition according to claim 13, wherein it further comprises at least one accelerating agent and/or at least one curing agent, preferably said curable composition comprises an amine-type curing agent, more preferentially the curing agent is isophorone diamine.

15. The curable composition according to claim 14, wherein the accelerating agents and/or curing agents are directly incorporated into the epoxy prepolymer composition.

16. The curable composition according to claim 15, wherein the accelerating agents and/or the curing agents are packaged separately from the epoxy prepolymer composition.

17. A polyepoxide obtained by curing the curable composition according to claim 14.

18. A composite, coating or adhesive material comprising the polyepoxide according to claim 17.
